(19) United States
(12) Patent Application Publication
(10) Pub. No.: US 2007/0253215 A1
(43) Pub. Date: Nov. 1, 2007

(54) LIGHT SOURCE APPARATUS FOR ENDOSCOPE SYSTEM

(75) Inventors: Tadashi TAKAHASHI, Saitama (JP); Nobuhiro TANI, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/736,776

(22) Filed: Apr. 18, 2007

(30) Foreign Application Priority Data
Apr. 27, 2006 (JP) 2006-123651

(51) Int. Cl. A61B 1/06

(52) U.S. Cl. 362/574

(57) ABSTRACT

A light source apparatus for an endoscope system comprises a light source, a light reduction unit, and a light guide. The light reduction unit has a light-passing area that is exposed to light from the light source. The light-passing area has a transmittance that is variable. The light guide illuminates an incident light through the light reduction unit, to a photographing subject. The transmittance is set based on a quantity of light emitted from the light source that varies with respect to a time of use of the light source.

### BACKGROUND OF THE INVENTION

1. Field of the Invention

The present invention relates to an endoscope system and in particular, relates to a light source apparatus for the endoscope system that adjusts a quantity of light.

2. Description of the Related Art

An apparatus that adjusts a quantity of light provided by the light source apparatus for the endoscope system, is proposed.

Japanese unexamined patent publication (KOKAI) No. 2000-201892 discloses a light source apparatus for an endoscope system that decreases a quantity of light when a quantity of light is manually adjusted and when a quantity of light over a permissible range is radiated.

However, a change in the quantity of light corresponding to the time the light source is in use is not considered.

### SUMMARY OF THE INVENTION

Therefore, an object of the present invention is to provide a light source apparatus for an endoscope system that can adjust a quantity of light provided, considering the occurrence of a variation in the quantity of light with respect to the amount of time the light source is in use.

According to the present invention, a light source apparatus for an endoscope system comprises a light source, a light reduction unit, and a light guide. The light reduction unit has a light-passing area that is exposed to light from the light source. The light-passing area has a transmittance that is variable. The light guide illuminates an incident light through the light reduction unit, to a photographing subject. The transmittance is set based on a quantity of light emitted from the light source that varies with respect to a time of use of the light source.

### BRIEF DESCRIPTION OF THE DRAWINGS

The objects and advantages of the present invention will be better understood from the following description, with reference to the accompanying drawings in which:

FIG. 1 is a construction diagram of the endoscope system;

FIG. 2 is a construction diagram of the light reduction board;

FIG. 3 is a graph that shows a sample of an actual change in the quantity of light emitted from the light source with respect to the time in use;

FIG. 4 is a graph that shows a change in the quantity of light emitted from the light source with respect to time, modeled from the actual change in the quantity of light in FIG. 3;

FIG. 5 is a sample part of the square-shaped mesh structure through which light passes;

FIG. 6 is a sample part of the slit-shaped mesh structure through which light passes;

FIG. 7 is a sample part of the round-shaped mesh structure through which light passes;

FIG. 8 is a table that shows a relationship between the time in use, the quantity of light, and the transmittance when the optimum value agrees with the regular quantity of light;

FIG. 9 is a table that shows a relationship between the time in use, the quantity of light, and the transmittance when the optimum value does not agree with the regular quantity of light; and

FIG. 10 is a table that shows a relationship between the light quantity level and the light quantity ratio.

### DESCRIPTION OF THE PREFERRED EMBODIMENTS

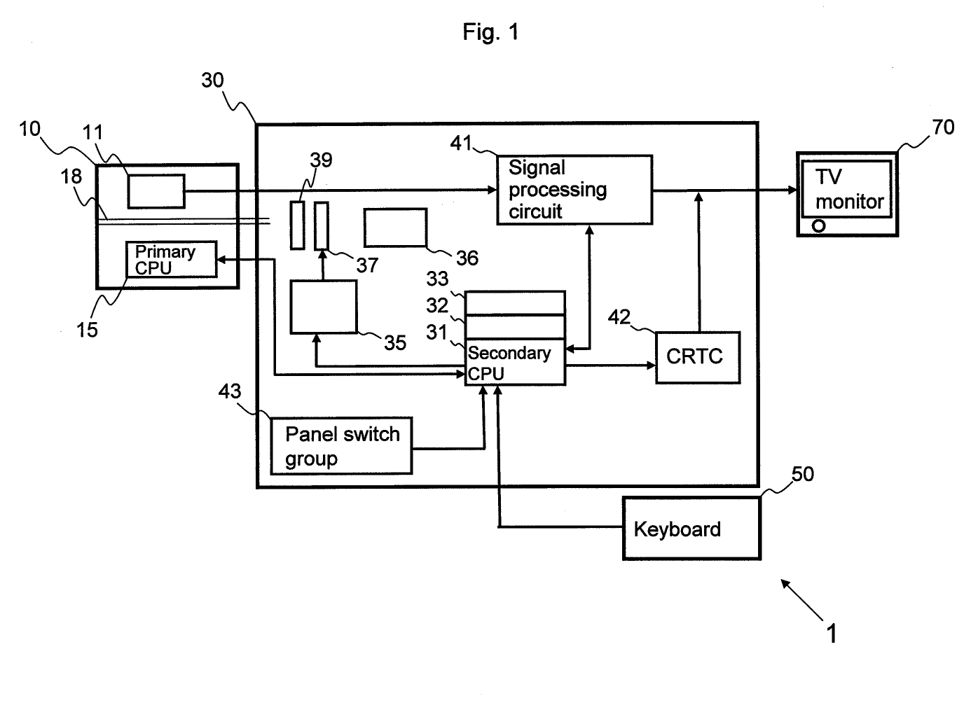

The present invention is described below with reference to the embodiments shown in the drawings. As shown in FIG. 1, an endoscope system in a first embodiment of the present invention is an electric endoscope apparatus that comprises an electric scope 10, a video processor 30, a keyboard 50, and a TV monitor 70.

The electric scope 10 includes an objective optical system (not depicted) and an imaging sensor 11 etc. at the small tipped-end of the electric scope 10. The small tipped-end of the electric scope 10 is inserted into an interior hollow of an organ where a photographic subject is imaged.

The electric scope 10 has an imaging sensor that is a CCD etc., and a primary CPU 15 (see FIG. 1). The electric scope 10 is connected to the video processor 30 through a connecting part (not depicted).

An image signal that is obtained by imaging at the imaging sensor 11 is output to the video processor 30 after undergoing a signal processing operation.

The primary CPU 15 is a one-tip microcomputer that controls each part of the electric scope 10 and performs a serial communication with a secondary CPU 31 of the video processor 30.

The video processor 30 has a secondary CPU 31, a memory 32, an RTC (Real Time Clock) 33, a motor 35, a light source 36 that is a lamp etc., a light reduction board (a neutral density board) 37, a signal processing circuit 41, a CRTC (CRT controller) 42, and a panel switch group 43.

The video processor 30 converts the image signal of the photographic subject that is imaged by the electric scope 10 to a video signal that can be displayed on the TV monitor 70. The video processor 30 illuminates the photographing subject through the small tipped-end of the electric scope 10. The light from the light source 36 is radiated to the photographing subject from the small tipped-end (a radiating end part) of the electric scope 10 through a light guide 18.

The secondary CPU 31 is a one-tip microcomputer that controls each part of the video processor 30, and performs a serial communication with the primary CPU 15 of the electric scope 10.

When a key of the keyboard 50 is operated, the secondary CPU 31 performs processing operations corresponding to the operation.

When a switch of the panel switch group 43 is operated, the secondary CPU 31 performs processing operations corresponding to the operation.

The secondary CPU 31 reads the time and date from the RTC 33, and indicates the time and date on the TV monitor 70 through the CRTC 42.

The secondary CPU 31 indicates the name, age, and sex of a patient, and the name of a doctor etc. on the TV monitor 70, through the CRTC 42.

The memory 32 is a non-volatile memory (EEPROM etc.) that is used for storing set values of each part of the video processor 30, and is connected to the secondary CPU 31. The memory 32 is also used for counting a time of use for the light source 36, which is described later.

In the embodiment, the time of use means a cumulative time of use for one light source (lamp).

The signal processing circuit 41 converts the image signal output from the electric scope 10 to the video signal displayed on the TV monitor 70.

The signal processing circuit 41 outputs a luminance signal based on the image signal to the secondary CPU 31. The secondary CPU 31 controls an aperture control circuit based on the luminance signal, changes an aperture degree (value) of an aperture 39, and adjusts a quantity of light that is transmitted from the light source 36 through the light reduction board 37 before entering the light guide 18 (an auto light control operation). Further, the aperture degree of the aperture 39 may be manually adjusted (a manual light control operation).

In the first embodiment, the light reduction board 37 is arranged between the light source 36 and the aperture 39. However, the light reduction board 37 may be arranged between the aperture 39 and the incident end of the light guide 18. In this case, when the aperture 39 is used in the light control operation, a quantity of light emanating from the light source 36 (Q₀ etc.), which is described later, is defined as a quantity of light entering the light reduction board 37 on the supposition that there is no aperture.

The light reduction board 37 is rotated by a motor 35 (a stepper motor) that is controlled by the secondary CPU 31.

The light reduction board 37 is disk-shaped with the light-passing area composed of a mesh structure, for example a wire netting etc. The light passing area of the light reduction board faces in the direction of the light source 36 so that light emitted from light source 36 is incident upon a portion of the mesh structure.

The mesh structure of the light-passing area of the light reduction board 37 is heterogeneous, with different parts (the meshed areas) transmitting different degrees of radiated light. The meshed areas are arranged sequentially from 0% transmittance to 100% transmittance.

The specific area of the mesh structure exposed to the light source 36 varies with the rotating movement of the motor 35, so that the transmittance of the meshed area exposed to the radiated light is variable.

When the radiated light is incident upon the meshed area whose transmittance is 0%, light radiating from the light source 36 does not pass through the 0% meshed area.

When the radiated light is incident upon the meshed area whose transmittance is 50%, only 50% of the light radiating from the light source 36 passes through the 50% meshed area.

When the radiated light is incident upon the meshed area whose transmittance is 100%, all of the light radiating from the light source 36 passes through the 100% meshed area.

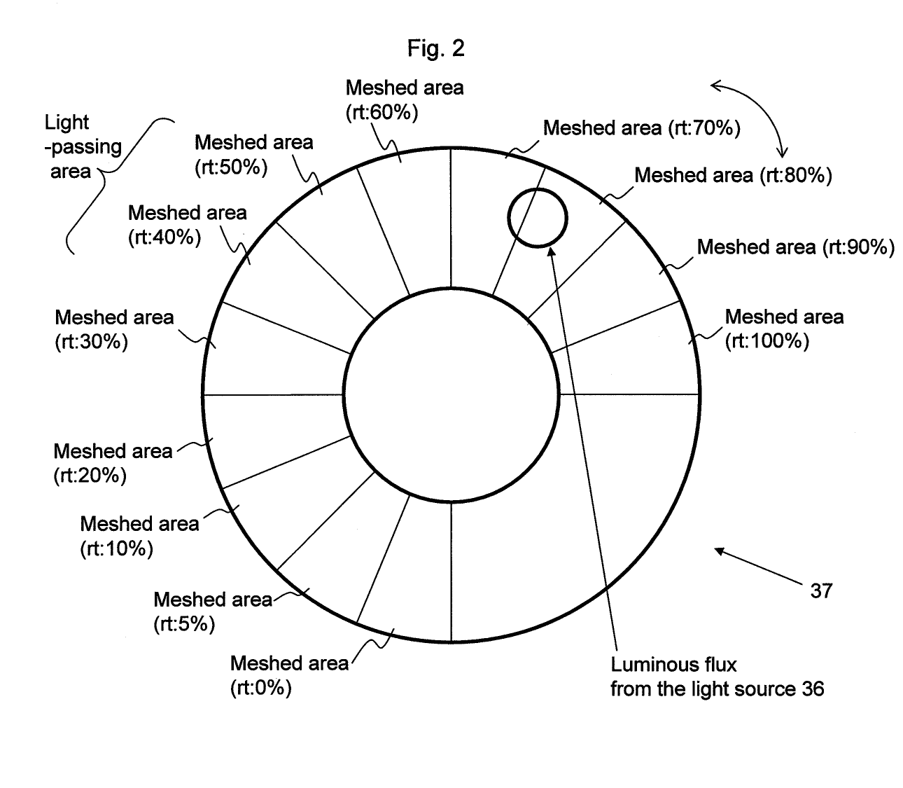

In FIG. 2, the radiated light from the light source 36 is incident upon an area between the 70% meshed area and the 80% meshed area, so that approximately 78% of the radiated light from the light source 36 passes through the light reduction board 37 in this configuration.

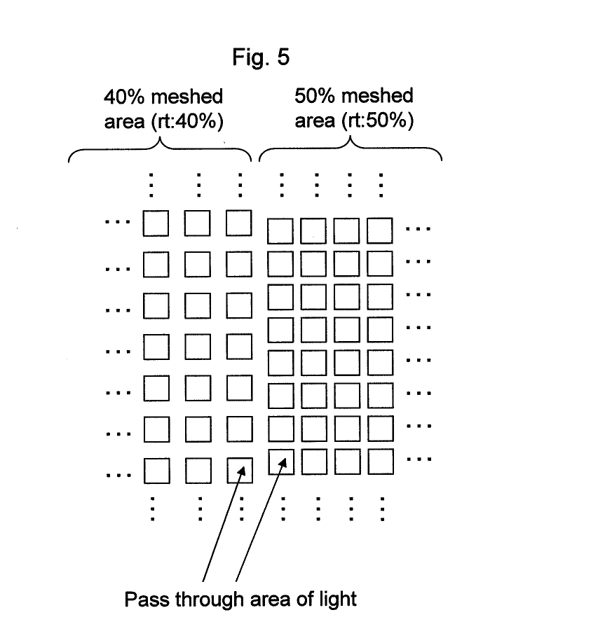

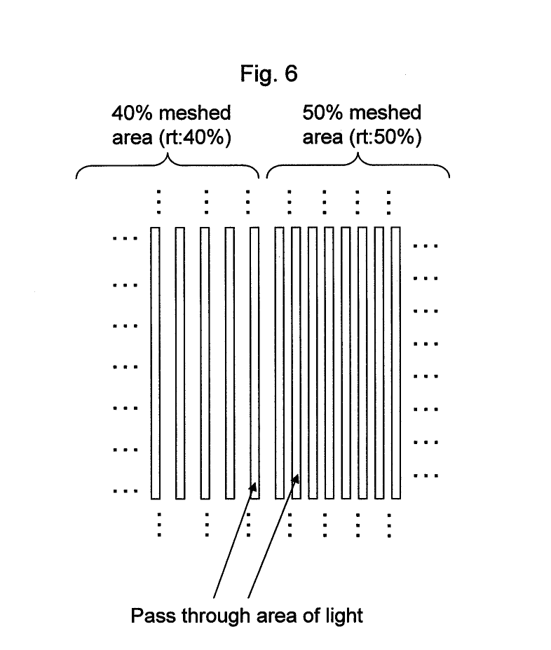

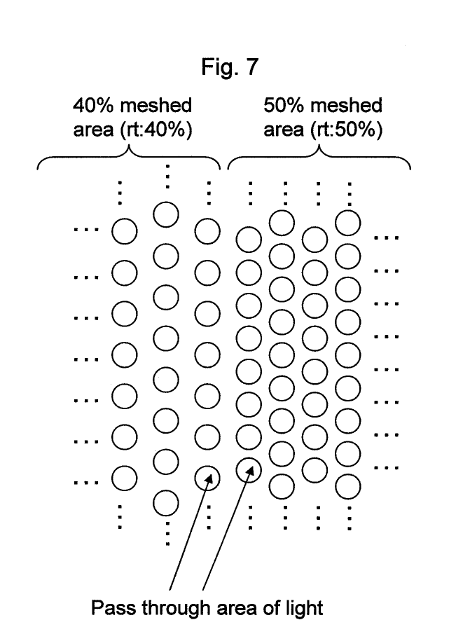

For example, one part of the light-passing area of the light reduction board 37 depicted in FIG. 5 has a transmittance of 40% (the 40% meshed area) and the other part has a transmittance of 50% (the 50% meshed area). FIG. 5 also indicates a square-shaped mesh structure through which the incident light passes, however, the mesh structure through which incident light passes may also be of a slit-shaped mesh structure (see FIG. 6) or round-shaped mesh structure (see FIG. 7).

Further, each meshed area is arranged sequentially in such a manner where there is no clearly defined boundary or partition separating adjacent meshed areas. Therefore, transmittance can be continuously modified.

The transmittance rt(t) is calculated based on an initial reference value Q₀ of the quantity of light emitted from the light source 36 (the initial quantity of light generated immediately upon operation of the light source 36), a regular time Tc, a regular quantity of light Qc emitted from the light source 36 after the regular time Tc has elapsed, and a time of use t of the light source 36.

The transmittance rt(t) is set so that the quantity of light transmitted from the light reduction board 37 is equal to or less than a constant optimum value Qp. The less than condition occurs when the quantity of light emanating from the light source 36 decreases to an amount that is lower than the optimum value Qp corresponding to the respective time of use. In this situation, the transmittance rt(t) is set to the maximum value (100%) so that the quantity of light emanating from the light source 36 and the quantity of light transmitted from the light reduction board 37 are in agreement with each other.

In the first embodiment, a value indicating the quantity of light, such as the optimum value Qp etc., is shown as a relative value to the regular quantity of light Qc that has a reference value of 100.

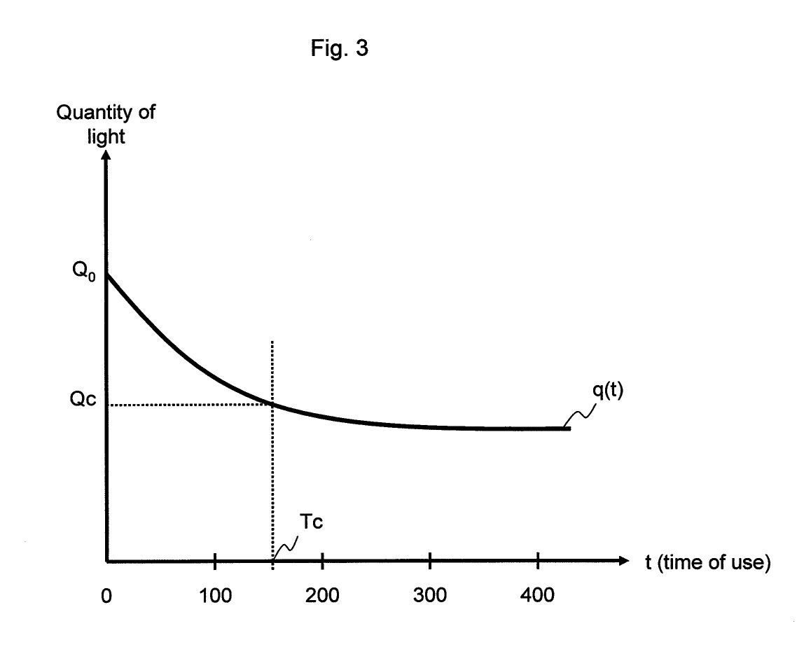

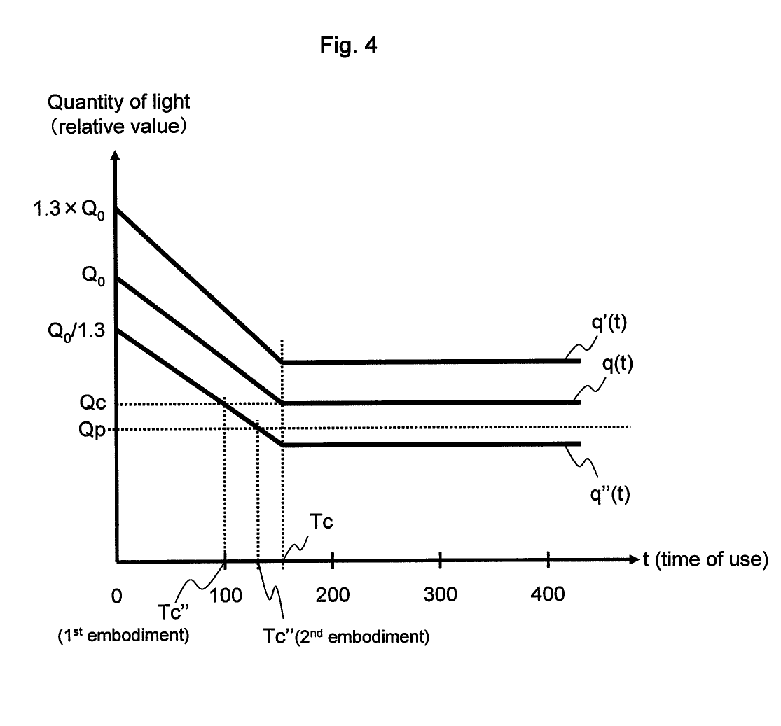

In the first embodiment, the optimum value Qp agrees with the regular quantity of light Qc (Qp=Qc) (see FIGS. 3 and 4). FIG. 3 shows an actual variation with respect to time of the quantity of light emitted from the light source. FIG. 4 shows a relative scenario modeled from the actual variation in FIG. 3, of a variation with respect to time of the quantity of light emitted from the light source, The quantity of light q(t) emitted from the light source 36 decreases linearly with respect to time of use t (q(t)={Tc×Q₀−(Q₀−Qc)×t}÷Tc, 0≦t≦Tc).

However, after a regular time Tc has elapsed (Tc≦t), the quantity of light q(t) emitted from the light source 36 becomes the regular value (q(t)=Qc, Tc≦t), or close thereto (see FIGS. 3 and 4).

Therefore, the light reduction board 37 is rotated to a position where the transmittance rt(t) is set to a variable governed by the equation (Qc×Tc)÷{Q₀×Tc−(Q₀−Qc)×t}×100(%), until the regular time Tc has elapsed (0≦t≦Tc).

The light reduction board 37 is fixed in the position where the transmittance rt(t) is set to the constant 100(%) (rt(t)=100) after the regular time Tc has elapsed (Tc≦t).

Therefore, a quantity of light (q(t)×rt(t)) transmitted from the light reduction board 37 is maintained at or near a constant value that is equal to both the regular quantity of light Qc and the optimum value Qp. Further, when the aperture degree of the aperture 39 is constant, a quantity of light that enters the incident end of the light guide 18 is maintained at a near-constant value.

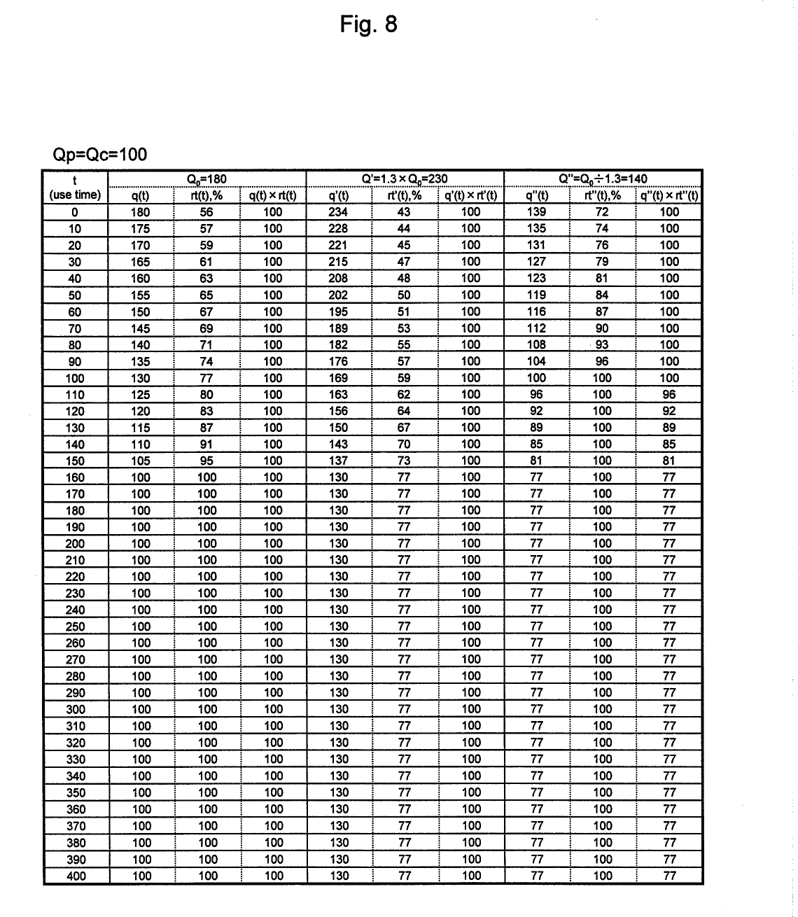

For example, when the initial reference value Q₀ is set to 180, the regular quantity of light Qc is set to 100, and the regular time Tc is set to 160; the transmittance rt(t) is set to a variable governed by the equation (100×160)÷{180×160−(180−100)×t}×100=160 ÷{18×16−0.8×t}×100 when (0≦t≦Tc), and the transmittance rt(t) is set to the constant 100 when (Tc≦t) (see the column of Q₀=180 in FIG. 8).

However, there is a case where variations in the light source 36 (lamp) cause the quantity of light emanating from the light source 36 that is used for the endoscope system 1 to vary substantially.

When the quantity of emanated light varies substantially on the high side (when the lamp of the light source 36 is brighter than a reference lamp), the quantity of light q′(t) emitted from the light source 36 decreases linearly with respect to the time of use t (q′(t)=m×{Tc×Q₀−(Q₀−Qc)×t}÷Tc, 0≦t≦Tc), so that after a regular time Tc has elapsed (Tc≦t), the quantity of light q′(t) emitted from the light source 36 approaches the regular value (q′(t)=m×Qc, Tc≦t) (see FIG. 4). The symbol “m” is a ratio of the initial high-side value Q′ (the initial quantity of light generated immediately upon operation of the light source 36) to the initial reference value Q₀ (m=Q′÷Q₀, m>1).

When the quantity of emanated light varies substantially on the low side (when the lamp of the light source 36 is darker than a reference lamp), the quantity of light q″(t) emitted from the light source 36 decreases linearly with respect to the time of use t (q″(t)=m×{Tc×Q₀−(Q₀−Qc)×t}÷Tc,0≦t≦Tc), so that after a regular time Tc has elapsed (Tc≦t), the quantity of light q″(t) emitted from the light source 36 approaches the regular value (q″(t)=m×Qc, Tc≦t) (see FIG. 4). The symbol “m” is a ratio of the initial low-side value Q″ (the initial quantity of light generated immediately upon operation of the light source 36) to the initial reference value Q₀ (m=Q″Q₀, m<1).

FIG. 4 shows the quantity of light q′(t) emitted from the light source 36 when the initial reference value Q₀ is set to 180, the ratio m is set to 1.3 (Q′=1.3×Q₀=230), and the quantity of light q″(t) emitted from the light source 36 when the initial reference value Q₀ is set to 180 and the ratio m is set to 0.77 (m=Q₀÷1.3=0.77, Q″=m×Q₀=140).

A transmittance rt′(t) for a quantity of light from the light source 36 that is considered to vary on the high side is calculated based on the initial reference value Q₀ of the quantity of light emitted from the light source 36, the regular time Tc, the regular quantity of light Qc emitted from the light source 36 after the regular time Tc has elapsed, the time of use t of the light source 36, and the ratio m (m=Q′÷Q₀, m>1).

A transmittance rt″(t) for a quantity of light from the light source 36 that is considered to vary on the low side is calculated based on the initial reference value Q₀ of the quantity of light emitted from the light source 36, the regular time Tc, the regular quantity of light Qc emitted from the light source 36 after the regular time Tc has elapsed, the time of use t of the light source 36, and the ratio m (m=Q″÷Q₀, m<1).

Specifically, under the condition of high side variation, the light reduction board 37 is rotated to a position where the transmittance rt′(t) is set to a variable governed by the equation (Qc×Tc)÷{m×Q₀×Tc−(m×Q₀−m×Qc)×t}×100(%), until the regular time Tc has elapsed (0≦t≦Tc). Under the same condition, the light reduction board 37 is rotated to a position where the transmittance rt′(t) is set to the value 1÷m×100(%) after the regular time Tc has elapsed (Tc≦t).

Therefore, a quantity of the light (q′(t)×rt′(t)) transmitted from the light reduction board 37 is maintained at or near a constant value that is equal to both the regular quantity of light Qc and the optimum value Qp. Further, when the aperture degree of the aperture 39 is constant, the quantity of light that enters the incident end of the light guide 18 is maintained at a near-constant value.

For example, when the initial reference value Q₀ is set to 180, the regular quantity of light Qc is set to 100, the regular time Tc is set to 160, and the ratio m is set to 1.3; the transmittance rt′(t) for a high side variance is set to a variable governed by the equation (100×160)÷{1.3×180×160−(1.3×180−1.3×100)×t}×100=160 ÷{1.3×18×16−1.3×0.8×t}×100 when (0≦t≦Tc), and the same transmittance rt′(t) is set to a constant 100 when (Tc≦t) (see the column of Q′=230 in FIG. 8).

Further, under the condition of low side variation, the light reduction board 37 is rotated to a position where the transmittance rt″(t) is either set to a variable governed by the equation (Qc×Tc)÷{m×Q₀×Tc−(m×Q₀−m×Qc)×t}×100(%), or is set to the fixed constant 100(%), until the regular time Tc has elapsed (0≦t≦Tc).

Specifically, when the value of (Qc×Tc)÷{m×Q₀×Tc−(m×Q₀−m×Qc)×t}×100 is less than or equal to 100, the quantity of light varies on the low side and the transmittance rt″ (t) is set to a variable governed by the equation (Qc×Tc)÷{m×Q₀×Tc−(m×Q₀−m×Qc)×t}×100(%). When the value of (Qc×Tc)÷{m×Q₀×Tc−(m×Q₀−m×Qc)×t}×100 is greater than 100, the transmittance rt″(t) is set to a constant 100(%).

After the regular time Tc has elapsed (Tc≦t), the light reduction board 37 is rotated to a fixed position where the transmittance rt″(t) is set to a constant 100(%) under the condition of low side variation.

Therefore, the quantity of light (q″(t)×rt″(t)) transmitted from the light reduction board 37 is maintained at or near a constant value that is equal to both the regular quantity of light Qc and the optimum value Qp, until time Tc″ when the value of (Qc×Tc)÷{m×Q₀×Tc−(m×Q₀−m×Qc)×t}×100 exceeds 100 (Tc″<Tc), under the condition of low side variation (see FIG. 4). Further, when the aperture degree of the aperture 39 is constant, the quantity of light that enters the incident end of the light guide 18 is maintained at a near-constant value.

The quantity of light (q″(t)×rt″(t)) transmitted from the light reduction board 37 is varied corresponding to the variations in the quantity of light q″(t) emitted from the light source 36 after time Tc″. Further, when the aperture degree of the aperture 39 is constant, the quantity of light that enters the incident end of the light guide 18 is modified corresponding to variations in the quantity of light q″(t) emitted by the light source 36.

For example, when the initial reference value Q₀ is set to 180, the regular quantity of light Qc is set to 100, the regular time Tc is set to 160, and the ratio m is set to 1.1.3=0.77; the low side transmittance rt″(t) is set to a variable governed by the equation (100×160)÷{0.77×180×160−(0.77×180−0.77×100)×t}×100=160÷{0.77×18×16−0.77×0.8×t}×100 when (0≦t≦Tc″), and the low side transmittance rt″ (t) is set to a constant 100 when (Tc″≦t) (see the column of Q″=140 in FIG. 8, Tc″=100).

The ratio m of the initial value Q^{(′,″)} of the varied quantity of light (the initial high side value of a quantity of light Q′ or the initial low side value of a quantity of light Q″) to the initial reference value Q₀ is based on the calculation of a relative initial value Qi of light emitted from the light source 36. The calculated relative initial value Qi is relative to a reference value of 100.

The calculation of the relative initial value Qi is based on lamp data of the light source 36 that has been measured in advance. Therefore, the ratio m is set by using the keyboard 50 etc. to input the calculated relative initial value Qi (m=Qi÷100=Q′÷Q₀ or Q″÷Q₀), when the light source (lamp) 36 that is used for the endoscope system 1 is changed.

The calculated relative initial value Qi is generally set between 70 and 140. When the light source (lamp) 36 is changed, the time of use t is reset to 0.

When the light source 36 is set to the on state, the secondary CPU 31 calculates the value of the ratio m based on the input value of the calculated relative initial value Qi during an initial setting operation of the secondary CPU 31, the transmittance rt(t) of the light reduction board 37, and the rotated position of the light reduction board 37. The secondary CPU 31 also drives the motor 35 to rotate the light reduction board 37 to the position corresponding to the calculated transmittance rt(t).

The light transmitted from the light reduction board 37 can be used as an illuminating light that is a quantity of light controlled either manually or automatically by the electrical shutter. In theses cases, the light transmitted from the light reduction board 37 is maintained at a near-constant value so that a heat infection caused either by radiation from the objective light emitted from the electric scope 10, or conduction from the incident end of the light guide 18, can be prevented.

Further, the quantity of light is adjusted near the light source 36 in consideration of the location of the variation, so that a light source 36 with a sizable variation can be used.

The measurement of the time of use t of the light source 36 is performed by the secondary CPU 31 with the use of a timer function. Specifically, the secondary CPU 31 performs an interruption process in 6-minute time intervals during the time in which the light source 36 is illuminated, so that the secondary CPU 31 adds 1 point to a counter ct in the memory 32 every six minutes. When the light source (lamp) 36 is changed, the value of the counter ct is reset to 0.

According to these operations, only 1 point is added to the value of the counter ct in a 6-minute time interval, so that the time of use t of the light source 36 is shown by 6×ct (minutes)=ct÷10 (hours).

For example, in the case that the value of the counter ct is 234, the use time t is equal to 6×234 (minutes)=23.4 (hours).

However, another method of counting the time of use t may be used.

Next, the second embodiment is explained (see FIG. 4). Constructions of the endoscope system 1 in the second embodiment are the same as those in the first embodiment. In the first embodiment, it is explained that the optimum value Qp is equal to the regular quantity of light Qc (Qp=Qc). However, in the second embodiment, the optimum value Qp is not equal to the regular quantity of light Qc. The different points from the first embodiment are explained as follows.

At first, the case involving the endoscope system 1 which uses a light source 36 that emits a quantity of light, that does not experience substantial variations caused by the variability of the light source 36 (lamp), is explained.

The light reduction board 37 is either rotated to a position where the transmittance rt(t) is set to a variable that is governed by the equation (Qp×Tc)÷{Q₀×Tc−(Q₀−Qc)×t}×100(%), or is fixed where the transmittance rt(t) is set to a constant 100(%), until when the regular time Tc has elapsed (0≦t≦Tc).

Specifically, when the value of (Qp×Tc)÷{Q₀×Tc−(Q₀−Qc)×t}×100 is equal to or less than 100, the transmittance rt(t) is set to a variable that is governed by the equation (Qp×Tc)÷{Q₀×Tc−(Q₀−Qc)×t}×100(%). When the value of (Qp×Tc)÷{Q₀×Tc−(Q₀−Qc)×t}×100 is greater than 100, the transmittance rt(t) is set to a constant 100 (%).

The light reduction board 37 is rotated to a position where the transmittance rt(t) is set to a variable that is equal to Qp÷Qc×100(%) or is fixed where the transmittance rt(t) is set to a constant 100(%), after the regular time Tc has elapsed (Tc≦t).

Specifically, when the value of Qp÷Qc×100 is equal to or less than 100, the transmittance rt(t) is set to a variable that is equal to Qp÷Qc×100(%). When the value of Qp÷Qc×100 is greater than 100, the transmittance rt(t) is set to a constant 100(%).

Therefore, the quantity of light (q(t)×rt(t)) transmitted from the light reduction board 37 is maintained at a near-constant value equal to the optimum value Qp, until time Tc″ when the value of (Qp×Tc)÷{Q₀×Tc−(Q₀−Qc)×t}×100 exceeds 100 (Tc″<Tc not depicted). Further, when the aperture degree of the aperture 39 is constant, the quantity of light that enters the incident end of the light guide 18 is maintained at a near-constant value.

The quantity of light (q(t)×rt(t)) transmitted from the light reduction board 37 is modified corresponding to variations in the quantity of light q(t) emitted by the light source 36 after time Tc″. Further, when the aperture degree of the aperture 39 is constant, the quantity of light that enters the incident end of the light guide 18 is modified corresponding to variations in the quantity of light q(t) emitted by the light source 36.

However, the value of (Qp×Tc)÷{Q₀×Tc−(Q₀−Qc)×t}×100 generally does not exceed 100, so that the quantity of light (q(t)×rt(t)) transmitted from the light reduction board 37 is maintained at a near-constant value equal to the optimum value Qp, based on the value of the regular quantity of light Qc and the optimum value Qp (ex. Qc≧QP).

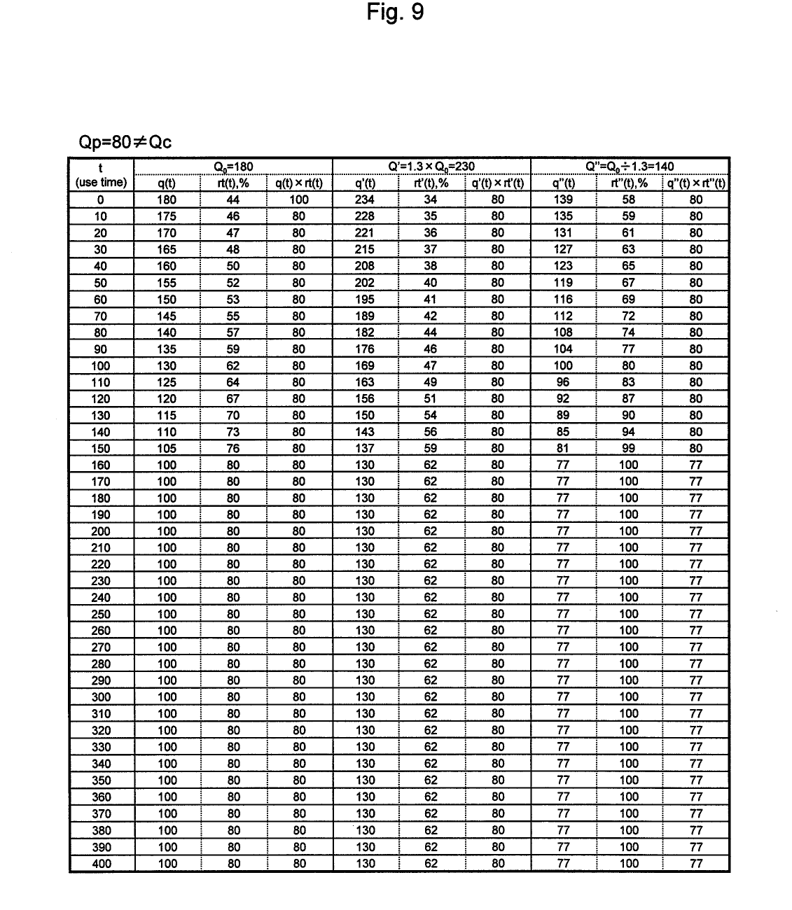

For example, when the initial reference value Q₀ is set to 180, the regular quantity of light Qc is set to 100, the regular time Tc is set to 160, and the optimum value Qp is set to 80; the transmittance rt(t) is set to a variable governed by the equation (80×160)÷{180×160−(180−100)×t}×100 when (0≦t≦Tc), and the transmittance rt(t) is set to a constant equal to 80÷100×100=80 when (Tc≦t) (see the column of Q₀=180 in FIG. 9).

Next, the case involving a light source 36 that emits a quantity of light that varies substantially on the high side of a reference value (when the lamp of the light source 36 is brighter than a reference lamp) is explained.

When a high side variation occurs, the light reduction board 37 is either rotated to a position where the transmittance rt′(t) is set to a variable that is governed by the equation (Qp×Tc)÷{m×Q₀×Tc−(m×Q₀−m×Qc)×t}×100(%), or fixed where the transmittance rt′(t) is set to a constant 100(%), until the regular time Tc has elapsed (0≦t≦Tc).

Specifically, under the condition of a high side variation, when the value of (Qp×Tc)÷{m×Q₀×Tc−(m×Q₀−m×Qc)×t}×100 is equal to or less than 100, the transmittance rt′(t) is set to a variable that is governed by the equation (Qp×Tc)÷{m×Q₀×Tc−(m×Q₀−m×Qc)×t}×100(%). When the value of the equation (Qp×Tc)÷{m×Q₀×Tc−(m×Q₀−m×Qc)×t}×100 is greater than 100, the transmittance rt′(t) is set to a constant 100(%).

The symbol “m” is a ratio of the initial high-side value Q′ (the initial quantity of light generated immediately upon operation of the light source 36) to the initial reference value Q₀(m=Q′÷Q₀, m>1).

When a high side variation occurs, the light reduction board 37 is either rotated to a position where the transmittance rt′(t) is set to a variable that is governed by the equation Qp÷(m×Qc)×100(%), or fixed where the transmittance rt′(t) is set to a constant 100(%), after the regular time Tc has elapsed (Tc≦t).

Specifically, under the condition of a high side variation, when the value of Qp÷(m×Qc)×100 is equal to or less than 100, the transmittance rt′(t) is set to a variable that is governed by the equation Qp÷(m×Qc)×100(%). When the value of Qp÷(m×Qc)×100 is greater than 100, the transmittance rt′(t) is set to a constant 100(%).

Therefore, the quantity of light (q′(t)×rt′(t)) transmitted from the light reduction board 37 is maintained at a near-constant value that is equal to the optimum value Qp, until time Tc″ when the value of (Qp×Tc)÷{m×Q₀×Tc−(m×Q₀−m×Qc)×t}×100 exceeds 100 (Tc″<Tc not depicted). Further, when the aperture degree of the aperture 39 is constant, the quantity of light that enters the incident end of the light guide 18 is maintained at a near-constant value.

The quantity of light (q′(t)×rt′(t)) transmitted from the light reduction board 37 is modified according to variations in the quantity of light q′(t) emitted from the light source 36 after time Tc″. Further, when the aperture degree of the aperture 39 is constant, the quantity of light that enters the incident end of the light guide 18 is modified according to variations in the quantity of light q′(t) emitted by the light source 36.

Under the condition of a high side variation, the value of (Qp×Tc)÷{m×Q₀×Tc−(m×Q₀−m×Qc)×t}×100 generally does not exceed 100, so that the quantity of light (q′(t)×rt′(t)) transmitted from the light reduction board 37 is maintained at a near-constant value equal to the optimum value Qp, based on the value of the regular quantity of light Qc, the optimum value Qp, and the value of the ratio m, etc.

For example, when the initial reference value Q₀ is set to 180, the regular quantity of light Qc is set to 100, the regular time Tc is set to 160, the ratio m is set to 1.3, and the optimum value Qp is set to 80; the transmittance rt′(t) based on a high side variation is set to a variable that is equal to (80×160)÷{1.3×180×160−(1.3×180−1.3×100)×t}×100 when (0≦t≦Tc), and set to a constant that is equal to 80÷(1.3×100)×100 when (Tc÷t) (see the column of Q′=230 in FIG. 9).

Next, the case involving a light source 36 that emits a quantity of light that varies substantially on the low side of a reference value (when the lamp of the light source 36 is darker than a reference lamp), is explained.

When a low side variation occurs, the light reduction board 37 is either rotated to a position where the transmittance rt″(t) is set to a variable that is governed by the equation (Qp×Tc)÷{m×Q₀×Tc−(m×Q₀−m×Qc)×t}×100(%), or fixed where the transmittance rt″(t) is set to constant 100(%), until the regular time Tc has elapsed (0≦t≦Tc).

Specifically, under the condition of a high side variation, when the value of (Qp×Tc)÷{m×Q₀×Tc−(m×Q₀−m×Qc)×t}×100 is equal to or less than 100, the transmittance rt″(t) is set to a variable that is governed by the equation (Qp×Tc)÷{m×Q₀×Tc−(m×Q₀−m×Qc)×t}×100(%). When the value of the equation (Qp×Tc)÷{m×Q₀×Tc−(m×Q₀−m×Qc)×t}×100 is greater than 100, the transmittance rt″(t) is set to a constant 100(%).

The symbol “m” is a ratio of the initial low-side value Q″ (the initial quantity of light generated immediately upon operation of the light source 36) to the initial reference value Q₀(m=Q″÷Q₀, m<1).

When a low side variation occurs, the light reduction board 37 is either rotated to a position where the transmittance rt″(t) is set to a variable that governed by the equation Qp÷(m×Qc)×100(%), or fixed where the transmittance rt″(t) is set to a constant 100(%), after the regular time Tc has elapsed (Tc≦t).

Specifically, under the condition of a low side variation, when the value of Qp÷(m×Qc)×100 is equal to or less than 100, the transmittance rt″(t) is set to a variable that is governed by the equation Qp÷(m×Qc)×100(%). When the value of Qp÷(m×Qc)×100 is greater than 100, the transmittance rt″(t) is set to a constant 100(%).

Therefore, the quantity of light (q″(t)×rt″(t)) transmitted from the light reduction board 37 is maintained at a near-constant value that is equal to the optimum value Qp, until time Tc″ when the value of (Qp×Tc)÷{m×Q₀×Tc−(m×Q₀−m×Qc)×t}×100 exceeds 100 (Tc″<Tc see FIG. 4). Further, when the aperture degree of the aperture 39 is constant, the quantity of light that enters the incident end of the light guide 18 is maintained at a near-constant value.

The quantity of light (q″(t)××rt′(t)) transmitted from the light reduction board 37 is modified according to variations in the quantity of light q″(t) emitted from the light source 36 after time Tc″. Further, when the aperture degree of the aperture 39 is constant, the quantity of light that enters the incident end of the light guide 18 is modified according to variations in the quantity of light q″(t) emitted by the light source 36.

Under the condition of a low side variation, the value of (Qp×Tc)÷{m×Q₀×Tc−(m×Q₀−m×Qc)×t}×100 generally does not exceed 100, so that the quantity of light (q″(t)×rt″(t)) transmitted from the light reduction board 37 is maintained at a near-constant value equal to the optimum value Qp, based on the value of the regular quantity of light Qc, the optimum value Qp, and the value of the ratio m, etc.

For example, when the reference initial value Q₀ is set to 180, the regular quantity of light Qc is set to 100, the regular time Tc is set to 160, the ratio m is set to 1.1.3=0.77, and the optimum value Qp is set to 80; the transmittance rt″(t) based on a low side variation is set to a variable that is equal to (80×160)÷{0.77×180×160−(0.77×180−0.77×100)×t}×100 when (0≦t≦Tc), and set to a constant that is equal to 100 (80÷(0.77×100)×100=103, which is greater than 100, therefore 100 is set) when (Tc≦t) (see the column of Q″=140 in FIG. 9).

Next, the third embodiment is explained. In the first and second embodiments, the aperture 39 that is used for adjusting the quantity of light transmitted from the light reduction board 37 that enters the incident end of the light guide 18 (light control operation), is positioned between the light source 36 and the light guide 18. In the third embodiment the endoscope system 1 does not have the aperture 39, so an adjustment of the transmittance of the light reduction board 37 produces the same effect as the light control operation comprised of adjusting the aperture degree of the aperture 39. In other words, the light reduction board 37 has an aperture adjusting function. The difference between the third embodiment and the first and second embodiments is explained below.

In the third embodiment, the light reduction board 36 is rotated to a position based on a re-calculated transmittance pt(t) that is the product of a light quantity ratio p and the same transmittance rt(t) calculated in the first and second embodiments.

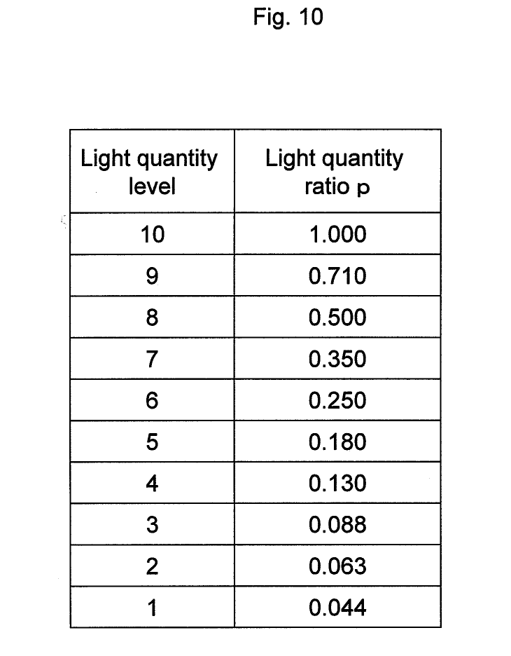

The light quantity ratio p is a ratio (aperture degree of aperture) corresponding to a light quantity level. The light quantity level is selected by the operation of the panel switch group 43 of the video processor 30 by an operator. FIG. 10 shows an example of a relationship between the light quantity level and the light quantity ratio.

Similarly, under the condition of a high side variation, the light reduction board 36 is rotated to a position based on a re-calculated transmittance pt′(t) for a high side variation that is the product of the light quantity ratio p and the same transmittance rt′(t) calculated for a high side variation in the first and second embodiments.

Further, similarly, under the condition of a low side variation, the light reduction board 36 is rotated to a position based on a re-calculated transmittance pt″(t) for a low side variation that is the product of the light quantity ratio p and the same transmittance rt″(t) calculated for a low side variation in the first and second embodiments.

Therefore, in the third embodiment, the aperture and the driving circuit for the aperture can be omitted.

Although the embodiments of the present invention have been described herein with reference to the accompanying drawings, obviously many modifications and changes may be made by those skilled in this art without departing from the scope of the invention.

The present disclosure relates to subject matter contained in Japanese Patent Application No. 2006-123651 (filed on Apr. 27, 2006) which is expressly incorporated herein by reference, in its entirety.

1. A light source apparatus for an endoscope system comprising:
a light source;
a light reduction unit that has a light-passing area that is exposed to light from said light source, said light-passing area having a transmittance that is variable; and
a light guide that illuminates an incident light through said light reduction unit, to a photographing subject;
said transmittance being set based on a quantity of light emitted from said light source that varies with respect to a time of use of said light source.

2. The light source apparatus according to claim 1, wherein said quantity of light emitted from said light source decreases with respect to time until a regular time has elapsed, and maintains a near-constant value after said regular time has elapsed.

3. The light source apparatus according to claim 2, wherein said transmittance is set where a quantity of light transmitted from said light reduction unit becomes a first quantity of light, when a quantity of light emitted from said light source is greater than said first quantity of light.

4. The light source apparatus according to claim 3, wherein said first quantity of light is equal to said near-constant value.

5. The light source apparatus according to claim 2, wherein said transmittance is set to a maximum value when a quantity of light emitted from said light source is not greater than a first quantity of light.

6. The light source apparatus according to claim 1, wherein the setting of said transmittance is based on a relative value between an initial quantity of light emitted from said light source installed in said endoscope system and an initial reference quantity of light emitted from a reference light source.

7. The light source apparatus according to claim 1, wherein said light reduction unit has an aperture adjusting function for a light control operation; and said transmittance is set corresponding to an aperture degree for said light control operation.

8. The light source apparatus according to claim 7, wherein said transmittance is set where a quantity of light transmitted from said light reduction unit becomes a first quantity of light, when a quantity of light emitted from said light source is greater than said first quantity of light; and said transmittance is set to a maximum value considering said aperture degree, when said quantity of light emitted from said light source is not greater than said first quantity of light.

9. The light source apparatus according to claim 1, wherein said light-passing area of said light reduction unit forms a mesh structure.
